(19) United States
(12) Patent Application Publication
(10) Pub. No.: US 2017/0000374 A1
(43) Pub. Date: Jan. 5, 2017

(54) PLANAR MAGNETIC RESONANCE SAFE CABLE FOR BIOPOTENTIAL MEASUREMENTS

(71) Applicant: KONINKLIJKE PHILIPS N.V., Eindhoven (NL)

(72) Inventors: Francis Patrick O'NEILL, KISSIMMEE, FL (US); Eduardo Mario REY, ORLANDO, FL (US); Donald Alan FORRER, ORLANDO, FL (US)

(21) Appl. No.: 15/102,545

(22) PCT Filed: Nov. 27, 2014
(86) PCT No.: PCT/IB2014/066379

§ 371 Date: Jun. 8, 2016

Related U.S. Application Data
(60) Provisional application No. 61/914,414, filed on Dec. 11, 2013.

(51) Int. Cl. A61B 5/0428; G01R 33/48; A61B 5/04

(52) U.S. Cl. CPC A61B 5/04286; A61B 5/04017; G01R 33/4808; A61B 2017/00911

(57) ABSTRACT

A magnetic resonance (MR) safe cable (10) includes four or more controlled resistance electrically conductive wires (12) disposed in a parallel planar configuration, and a stiff non-proton emitting substrate (14) which holds the four or more controlled resistance conductive wires in the parallel planar configuration. The wires (12) terminating at one end in connectors which electrically connect to ECG electrodes (64) which are attached to the subject. The cable is configured to extend the one end into an imaging region of an MR scanner (62) during imaging to carry ECG signals to associated equipment.

The following relates generally to magnetic resonance (MR) imaging. It finds particular application in conjunction with Electrocardiography (ECG) during MR imaging, and will be described with particular reference thereto. However, it will be understood that it also finds application in other usage scenarios and is not necessarily limited to the aforementioned application.

MR imaging often involves biopotential measurements such ECG monitoring during the imaging procedure. Patients can be monitored for vital signs with ECG or other monitors during MR procedures to ensure patient health. ECG monitors can be used in MR imaging as triggers or gates for image capture. For example, images can be triggered such that the heart of a subject is shown only in images in a certain phase or which compensates for heart motion artifacts in the image.

ECG monitoring uses electrodes affixed to the patient at different points on the body. The electrodes sense weak electrical signals from the body which indicate phases of the heart. The electrodes are attached to leads which transmits the sensed signals to an ECG monitor which analyses the signal and/or displays the signal visually. Each lead includes a conductive wire or trace which connects the electrode to the ECG monitor and transmits signals from the attached electrode to the ECG monitor. The number of electrodes and corresponding leads can vary which means the number of wires connecting electrodes of one patient to an ECG monitor can vary. The leads can also include leads for other biopotential measurements of the patient such as respiratory monitors and the like.

Magnetic Resonance scanners use strong magnetic fields for imaging the patient and the strong magnetic fields can induce electric currents in conductive wires such as ECG traces or wires. The magnetic fields of the scanner can induce currents on the ECG wires such that give potentially false heart rate readings or obscure ECG R-waves from waveform detection schemes. The wires are susceptible to triboelectric effects and are hyper-sensitive to patient movement. Cross-talk between wires can interfere with the transmitted signals. Looping of wires can add to the interference where one or more wires loop around in close proximity to the same or other lead wires. Discrete wires can be inconsistent and/or inaccurate with transmitting the ECG signals. Improperly selected materials of the wire, cabling and/or insulating material which emits protons can cause artifacts in images.

Care must be taken in selecting and configuring the materials of the cabling including the wires because of patient safety. Ferrous materials can become projectiles in the strong magnetic fields of scanners which can injure patients and/or healthcare workers. Induced currents on non-MR ECG cables in the magnetic field can cause heating of the wires which may subject the patient to burns.

Current approaches for MR cables typically use high resistance wires which are braided or twisted to reduce susceptibility to noise pickup from the MR scanner. The braided or twisted bundles of wire are then insulated. Some approaches then add a shielding to further minimize interference from the magnetic field. However, these approaches still contend with cross talk between the wires twisted or braided together and do not address problems caused with looping of wires. Additionally, the twisting or braiding of the wires, and adding components such as shielding adds to the cost of the cable.

The following discloses a new and improved planar magnetic resonance safe cable for biopotential measurements which addresses the above referenced issues, and others.

In accordance with one aspect, a magnetic resonance (MR) safe cable includes four or more controlled resistance electrically conductive wires disposed in a planar configuration and parallel to each other, and a stiff non-proton emitting substrate which holds the four or more controlled resistance conductive wires in the planar configuration and parallel to each other.

In accordance with another aspect, a method of using a magnetic resonance (MR) safe cable includes attaching at least four electrodes to a subject. The MR safe cable is disposed along the subject supported by the subject or a subject support, and the MR cable includes at least four controlled resistance conductive wires disposed in a parallel planar configuration. A connector attached to one of the corresponding connectors of a first end of the at least four controlled resistance conductive wires is connected to the electrodes. Connectors attached to a second end of the controlled resistance conductive wires are connected to a patient monitor. The four or more controlled resistance conductive wires in the planar and parallel configuration are held with a stiff non-proton emitting substrate of the MR safe cable which encases the controlled resistance wires in the parallel planar configuration.

In accordance with another aspect, a method of manufacturing a magnetic resonance (MR) safe cable includes stretching at least four controlled resistance electrically conductive wires parallel and in a plane, and coating the stretched at least four controlled resistance electrically conductive wires with a stiff foam jacket.

One advantage is a cable which operates in a strong magnetic field that is more reliable and repeatable for biopotential measurements.

Another advantage is in reducing variation in induction of interference currents in the electrode and resistive traces from the static and gradient magnetic fields.

Another advantage is in reducing triboelectric and microphonic effects by eliminating movement between resistive traces in the cable.

Another advantage resides in the increased likelihood of induced currents equally on each lead which can then be canceled out by a common mode filter.

Another advantage resides in patient safety in a strong magnetic field.

Another advantage resides in a stable, distributed, and high resistance trace.

Another advantage resides in a cable which does not cause artifacts in MR imaging.

Still further advantages will be appreciated to those of ordinary skill in the art upon reading and understanding the following detailed description.

The invention may take form in various components and arrangements of components, and in various steps and arrangement of steps. The drawings are only for purposes of illustrating the preferred embodiments and are not to be construed as limiting the invention.

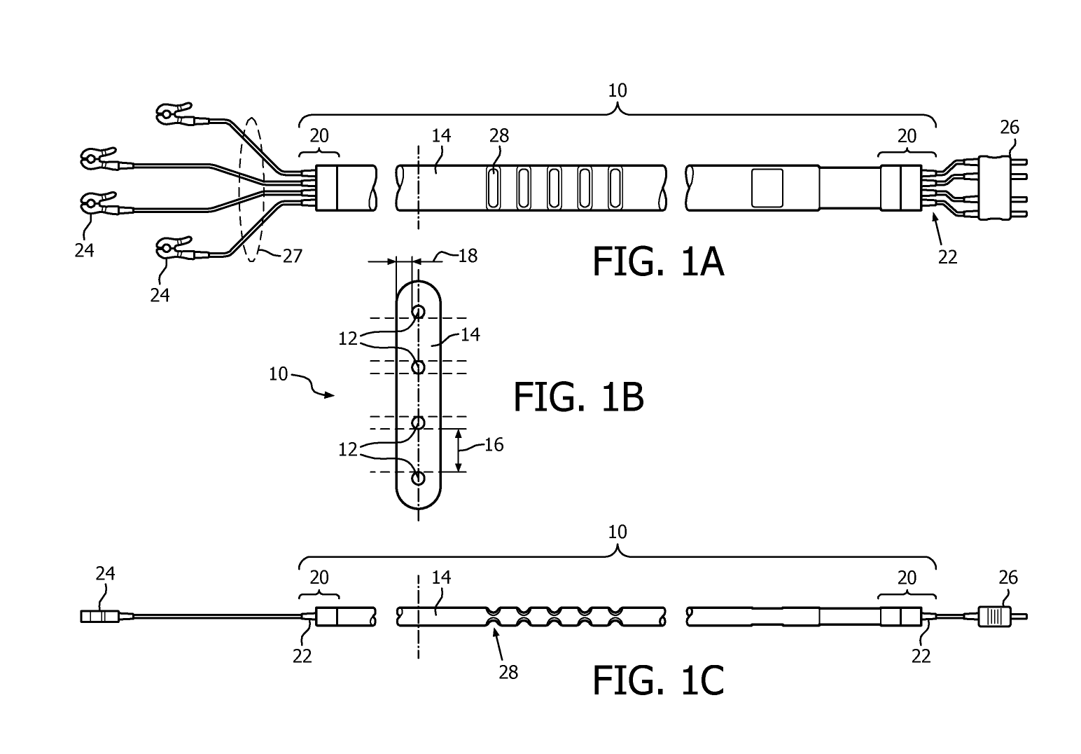

FIGS. 1A-1C schematically illustrates top, cross-sectional, and side views of an embodiment of a planar magnetic resonance safe cable for biopotential measurements.

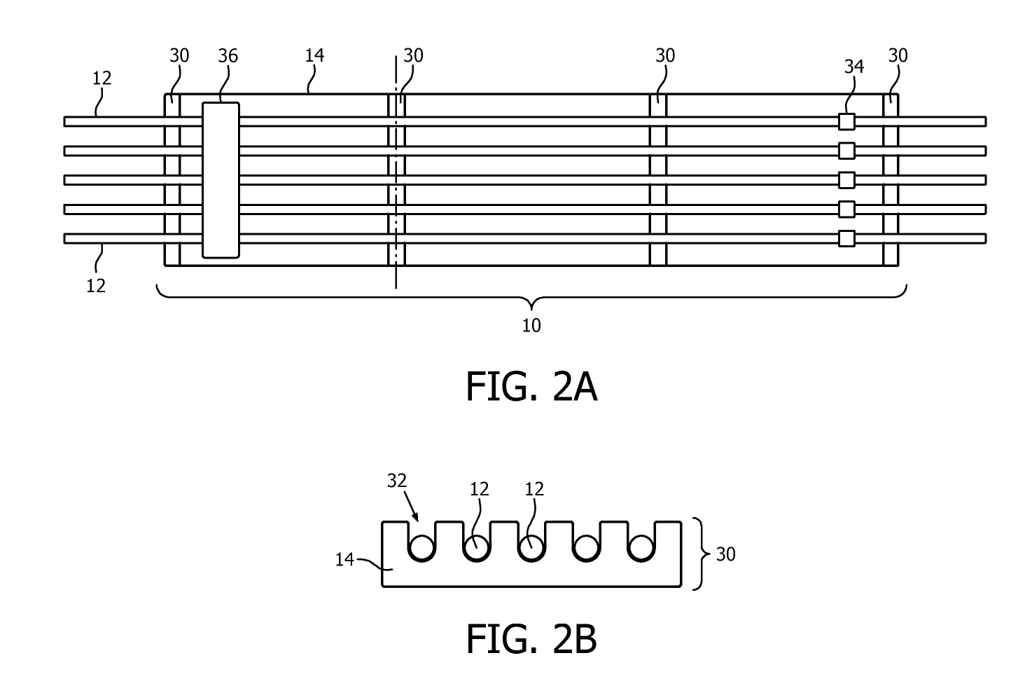

FIGS. 2A-2B schematically illustrates top and transverse views of another embodiment of the planar magnetic resonance safe cable for biopotential measurements.

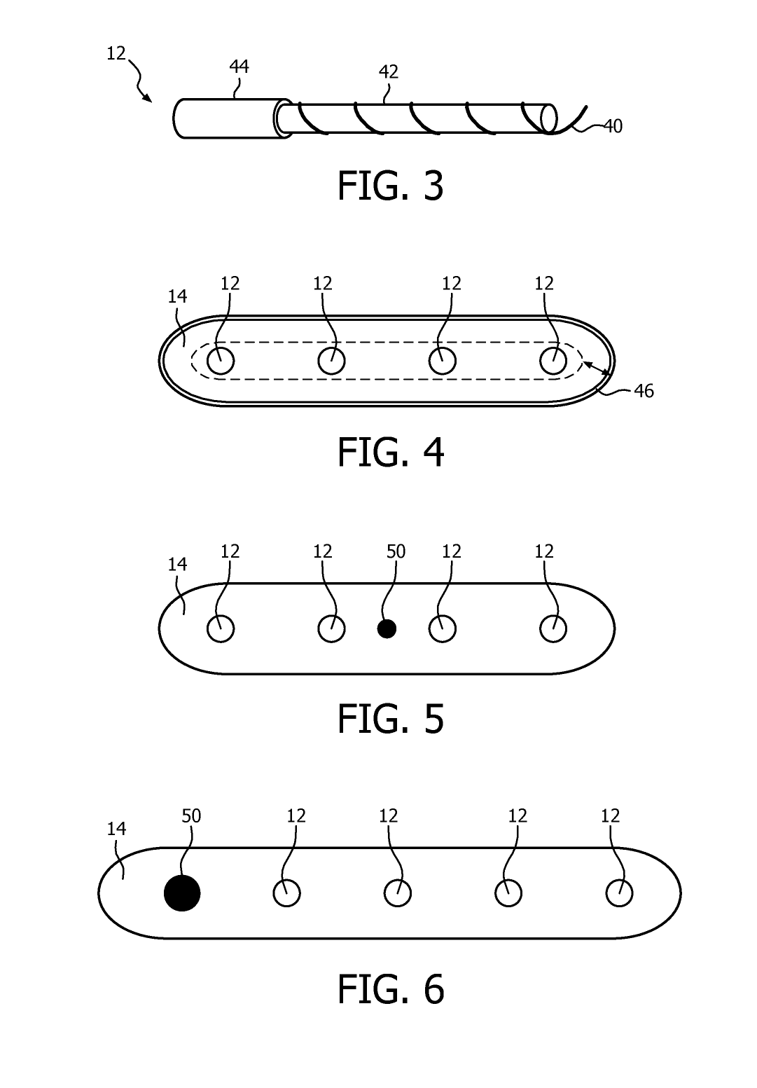

FIG. 3 schematically illustrates an embodiment of a controlled resistance electrically conductive wire.

FIG. 4 schematically illustrates another embodiment of the planar magnetic resonance safe cable with protective shielding.

FIG. 5 schematically illustrates another embodiment of the planar magnetic resonance safe cable with an additional component between controlled resistance electrically conductive wires.

FIG. 6 schematically illustrates another embodiment of the planar magnetic resonance safe cable in a cross section with an additional component adjacent the controlled resistance electrically conductive wires.

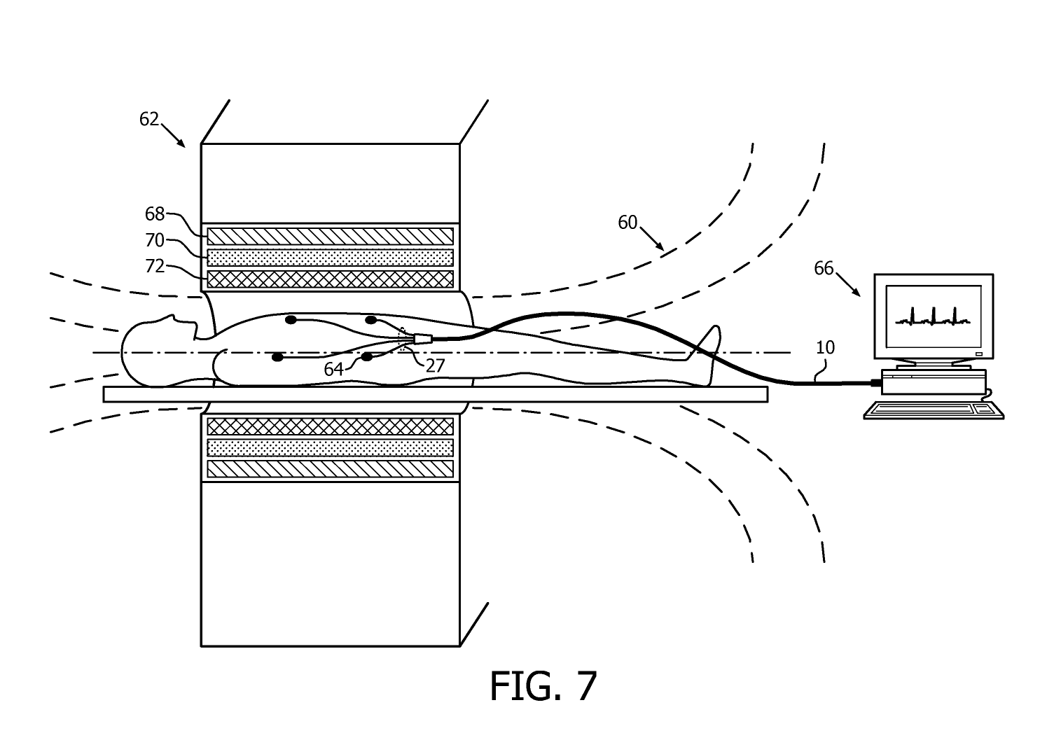

FIG. 7 schematically illustrates another embodiment of the planar magnetic resonance safe cable with a MR scanner and ECG monitor.

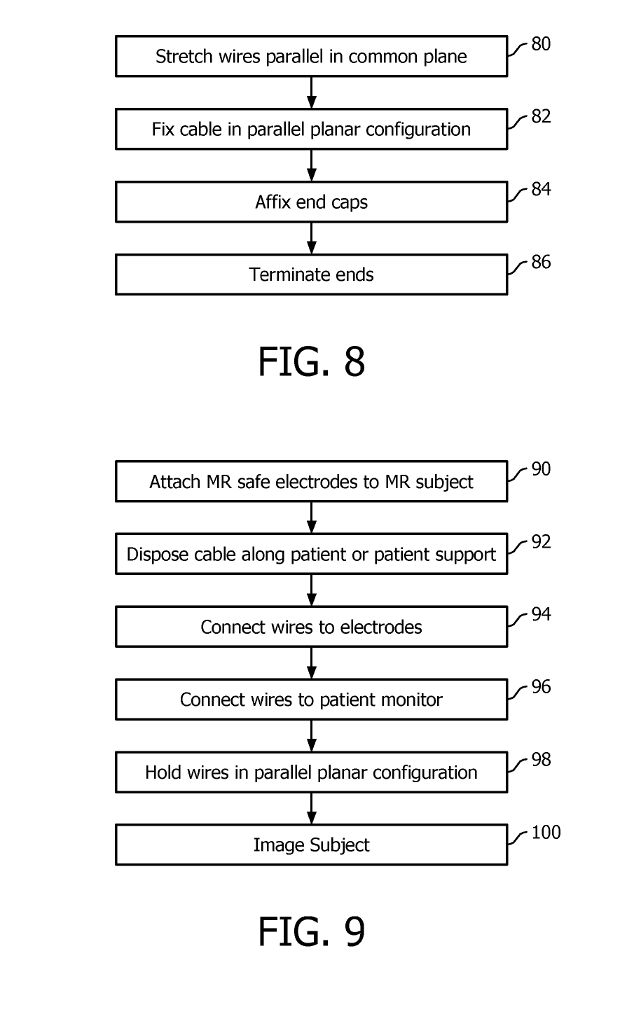

FIG. 8 flowcharts one method of manufacturing an embodiment of the planar magnetic resonance safe cable for biopotential measurements.

FIG. 9 flowcharts one method of using an embodiment of the planar magnetic resonance safe cable for biopotential measurements.

With reference to FIG. 1A, an embodiment of a planar magnetic resonance (MR) safe cable 10 for biopotential measurements is schematically illustrated in a top view. In FIG. 1B, the MR safe cable 10 is shown in a cross section view, and in FIG. 1C, the MR safe cable 10 is shown in a side view. The cable 10 is shown with four controlled resistance electrically conductive wires 12 disposed in a common plane and parallel to each other. The cable can be configured in other embodiments to hold any number of wires, e.g., 4 to 12, in the common plane and parallel to each other. The wires transmit physiological signals such as ECG signals in a strong magnetic field. The strong magnetic field can be a magnetic field of a magnetic resonance imaging (MM) scanner. The controlled resistance electrically conductive wires in one embodiment include a distance 16 of at least 4 mm between adjacent wires, but other distances are contemplated.

The cable 10 includes a stiff flat substrate 14 which holds the four or more controlled resistance conductive wires in the common plane and parallel to each other. The substrate is made of a non-proton emitting material. The stiff planar substrate can include a foam plastic jacket which surrounds and holds the conductive wires parallel and in the common plane. Holding the conductive wires parallel and in a common plane increases the likelihood that induced currents will be induced equally on equal wire and therefore can be filtered out more easily due to “common-mode”. The foam jacket thermally and electrically insulates the wires from contact with the patient. The foam in one embodiment includes at least 2.5 mm of foam 18 between each controlled resistance electrically conductive wire and an outside surface. The foam can include open or closed cell foam. The closed cell foam provides easier clean-up.

End caps 20 attached to each end of the stiff planar substrate can provide strain relief to the four or more controlled resistance electrically conductive wires and guide the wires into predetermine connections. For example, each wire can be supported with strain relief 22 between a connector and an end of the substrate or foam jacket. The end caps can provide a rigid surface for grasping the cable and fitting the connector. The end caps reduce overall wear on the cable. Connectors 24 disposed at one end of the cable connect, e.g. clip, each wire to a contact of an ECG electrode. Connectors 26 disposed at another end connect, e.g. plug, the wires to a patient monitor. The connectors can be keyed such as color coded, marked, shaped, and the like, which provide a healthcare practitioner easy identification of corresponding predetermined connections for each connector. The connectors can be individual connectors such as shown with individual leads 27 and the electrode connectors, or unitary connector such as shown with the patient monitor connectors. A unitary connector can include webbing which additionally provides strain relief, organization, and ordered connection. The leads 27 can be in a staggered configuration as shown with varying length of leads or in a straight configuration with equal length leads.

The cabling can include necking 28 in the foam jacket or substrate to facilitate storage. For example, the cable can be wound in a large coil and fastened with a temporary fastener for storage. Removing the fasteners allows the cable to extend or return to the planar position.

With reference to FIGS. 2A-2B, another embodiment of the planar magnetic resonance safe cable 10 for biopotential measurements is schematically illustrated. In FIG. 2A, the cable 10 is shown in a top view, and in FIG. 2B, the cable 10 is shown in a cross section view. As shown, the stiff planar substrate 14 holds five controlled resistance conductive wires 12 in a common plane and parallel to each other. The substrate 14 includes MR inert material or material that does not emit protons. The stiff substrate 14 includes a plurality of cable combs 30 spaced to hold the controlled resistance wires 12 in the common plane, with a fixed spacing and parallel to each other. Each comb 30 includes slots 32 and each slot holds one of the wires parallel to adjacent wires and the wires collectively in the common plane.

Each controlled resistance electrically conductive wire can include electric shock inhibiting components 34, such as discrete resistors, RF chokes, and the like. For example, a predetermined or variable resistor is spliced into each wire to control resistance of the wire. The wires 12 can individually or collectively include one or more additional components 36 such as a notch filter, low pass filter, integrated circuit, sensor, and the like. The filters can block currents at frequencies other than the frequencies with ECG signals.

The embodiment of FIGS. 2A and 2B can be integrated into the embodiment of FIG. 1A-1C such as using the cable combs during manufacture and integrating the substrate of FIGS. 2A and 2B into the foam jacket.

With reference to FIG. 3, an embodiment of a controlled resistance electrically conductive wire 12 is schematically illustrated. The controlled resistance electrically conductive wire can include fine high resistance wire 40 wound spirally around a non-conductive core 42. The fine high resistance wire 40 can include a nickel copper alloy. The controlled resistance electrically conductive wire 12 can include an insulating material cover 44. The non-conductive core 42 and the insulating material cover 44 are made of MR inert material.

With reference to FIG. 4, another embodiment of the planar magnetic resonance safe cable 10 is schematically illustrated in a cross section. The cable can include a protective shield 46, such as a non-ferrous foil or copper mesh, surrounding the controlled resistance electrically conductive wires 12. The protective shield further protects the wires 12 from electrical interference. The protective shield is disposed as an external covering as shown or integrated into the foam jacket as indicated by the dotted lines.

With reference to FIG. 5 another embodiment of the planar magnetic resonance safe cable 10 is schematically illustrated in a cross section with an additional optically or electrically conductive component 50, such as an optical fiber, antenna lead, sensor lead, integrated circuit, power supply lead, and the like. The conductive component can be located between the controlled resistance electrically conductive wires and/or as shown in FIG. 6 adjacent to the wires 12.

With reference to FIG. 7 another embodiment of the planar magnetic resonance safe cable 10 schematically illustrated in a magnetic field 60 of an MR scanner 62. The MR scanner 62 is shown in a cross section. The cable 10 is shown connected by leads 27 to electrodes 64 of a subject and to a patient monitor 66, such as an ECG monitor, respiratory monitor, and the like. The MR scanner generates a horizontal or vertical static B₀ field from a magnet coil 68. The MR scanner generates gradient magnetic or B₁ fields from gradient coils 70. The MR scanner induces resonance in tissues of the subject with a local and/or whole body RF coil 72. The cable 10 receives the sensed physiological signals from electrodes attached to the subject and transmits the physiological signals to the patient monitor in the presence of the magnetic and RF fields generated by the various coils.

The cable 10 holds the controlled resistance electrically conductive wires planar and parallel. The cable substrate forms a stiff jacket that resists looping and twisting. The substrate returns to the cable to a substantially straight cable run. The cable without force applied returns to a planar configuration, e.g. if placed on a flat surface will return to straight and flat.

With reference to FIG. 8, one method of manufacturing an embodiment of the planar magnetic resonance safe cable 10 for biopotential measurements is flowcharted. In a step 80 at least four controlled resistance electrically conductive wires 12 are stretched parallel and in a common plane. The wires are stretched perpendicular between two lines such as between two cable combs. The step can include stretching additional leads in the common plane such as optical fibers, antenna, power cables, and the like. The step can include adding discrete resistance components, integrated circuits, sensors, and the like.

In a step 82, a stiff foam jacket coats the stretched at least four controlled resistance electrically conductive wires. The stiff foam jacket holds or fixes the wires parallel and in the common plane. In one embodiment, the foam jacket is pressure molded. The cable combs can be integrated into the stiff foam jacket.

Affixing end caps to each end of the foam jacket provide strain relief in a step 84. The end caps can be integrated with the foam jacket or added as a separate step.

In a step 86, the least four controller resistance electrically conductive wires are terminated at a first end with electrode connectors and at a second end with patient monitor connectors. The step can include adding keys to the connectors or leads.

With reference to FIG. 9, one method of using an embodiment of the planar magnetic resonance safe cable 10 for biopotential measurements is flowcharted. In a step 90, at least four electrodes are attached to a subject.

In a step 92, the MR safe cable is disposed along the subject supported by the subject or a subject support. The MR safe cable includes the at least four controlled resistance conductive wires disposed in a parallel planar configuration. The step can include releasing the MR safe cable from a storage configuration to an operation configuration.

A connector attached to one of the corresponding connectors of a first end of the at least four controlled resistance conductive wires is connected to the electrodes in a step 94. Connectors attached to a second end of the controlled resistance conductive wires are connected to a patient monitor in a step 96.

With a stiff non-proton emitting or MR inert substrate of the MR safe cable which encases the controlled resistance wires in the parallel planar configuration, the four or more controlled resistance conductive wires are held in the planar and parallel configuration in a step 98. The wires transmit the ECG signals from the electrodes to the patient monitor for biopotential measurements. The stiff substrate can include the embodiments of the cable as described in reference to FIGS. 1A-1C, 2A-2B, 3-7, and combinations thereof.

In a step 100, the subject can be imaged with a magnetic resonance scanner and the connected MR safe cable. The step includes transmitting physiological signals such as ECG signals with the MR safe cable concurrently with the imaging which are analyzed and/or displayed. The imaging can include processing of the transmitted signals such as with triggered or gated imaging.

It is to be appreciated that in connection with the particular illustrative embodiments presented herein certain structural and/or function features are described as being incorporated in defined elements and/or components. However, it is contemplated that these features may, to the same or similar benefit, also likewise be incorporated in other elements and/or components where appropriate. It is also to be appreciated that different aspects of the exemplary embodiments may be selectively employed as appropriate to achieve other alternate embodiments suited for desired applications, the other alternate embodiments thereby realizing the respective advantages of the aspects incorporated therein.

For example, the lead wires 12 can be disposed in other relationships that resist looping, twisting, or otherwise moving relative to each other. In one example, the lead wires are disposed alternately in first and second adjacent parallel planes.

It is also to be appreciated that particular elements or components described herein may have their functionality suitably implemented via hardware, software, firmware or a combination thereof. Additionally, it is to be appreciated that certain elements described herein as incorporated together may under suitable circumstances be stand-alone elements or otherwise divided. Similarly, a plurality of particular functions described as being carried out by one particular element may be carried out by a plurality of distinct elements acting independently to carry out individual functions, or certain individual functions may be split-up and carried out by a plurality of distinct elements acting in concert. Alternately, some elements or components otherwise described and/or shown herein as distinct from one another may be physically or functionally combined where appropriate.

In short, the present specification has been set forth with reference to preferred embodiments. Obviously, modifications and alterations will occur to others upon reading and understanding the present specification. It is intended that the invention be construed as including all such modifications and alterations insofar as they come within the scope of the appended claims or the equivalents thereof. That is to say, it will be appreciated that various of the above-disclosed and other features and functions, or alternatives thereof, may be desirably combined into many other different systems or applications, and also that various presently unforeseen or unanticipated alternatives, modifications, variations or improvements therein may be subsequently made by those skilled in the art which are similarly intended to be encompassed by the following claims.

1. A magnetic resonance safe cable, comprising:
four or more electrically conductive wires disposed in a planar configuration and parallel to each other; and
a stiff non-proton emitting substrate which holds the four or more controlled resistance conductive wires in the planar configuration and parallel to each other;
wherein the stiff substrate includes a flat foam jacket which surrounds and holds the conductive wires parallel and in the common plane.

2. (canceled)

3. The MR safe cable according to claim 1, wherein the stiff substrate includes a plurality of longitudinally spaced cable combs spaced to hold the conductive wires in the planar configuration and parallel to each other, each comb including a plurality of slots configured to receive and anchor one of the wires in the parallel planar configuration.

4. The MR safe cable according to claim 1, wherein the substrate fixes the controlled resistance electrically conductive wires in the common plane separated by at least 4 mm.

5. (canceled)

6. The MR safe cable according to claim 1, wherein the flat foam jacket includes closed cell foam.

7. The MR safe cable according to claim 1, wherein the flat foam jacket includes at least 2.5 mm of foam between each conductive wire and an outside surface.

8. The MR safe cable according to claim 1, wherein each conductive wire includes high resistance wire wound spirally around a non-conductive core and covered with an insulating material.

9. The MR safe cable according to claim 1, wherein each conductive wire includes discrete resistance components disposed within the stiff substrate.

10. The MR safe cable according to claim 1, further including:
a protective shield surrounding the stiff substrate.

11. The MR safe cable according to claim 1, further including:
end caps attached to each end of the stiff substrate which provide strain relief to the four or more conductive wires and guide the conductive wires towards predetermined connections.

12. The MR safe cable according to claim 1, further including:
connectors disposed at one end of the cable which connect each wire to an ECG electrode; and
connectors disposed at another end of the cable which connect the wires to a patient monitor.

13. (canceled)

14. (canceled)

15. The MR safe cable according to claim 1, wherein the stiff substrate further houses:
at least one of an integrated circuit, a notch filter, low pass filter, a power supply lead, a sensor, and an optical fiber.

16. A method of using a magnetic resonance safe cable, comprising:
attaching at least four electrodes to a subject;
disposing the MR safe cable along the subject supported by the subject or a subject support, the MR cable including at least four conductive wires disposed in a parallel planar configuration and housed in a foam jacket;
connecting a connector attached to one of the corresponding connectors of a first end of the at least four conductive wires to the electrodes;
connecting connectors attached to a second end of the conductive wires to a patient monitor; and
with a stiff non-proton emitting substrate of the MR safe cable which encases the conductive wires in the parallel planar configuration, holding the four or more controlled resistance conductive wires in the planar and parallel configuration.

17. The method according to claim 16, further including:
imaging the subject with a magnetic resonance scanner with the MR safe cable disposed partially in an imaging region of the magnetic resonance scanner.

18. A method of manufacturing a magnetic resonance safe cable, comprising:
stretching at least four electrically conductive wires parallel and in a plane;
coating the stretched at least four electrically conductive wires with a stiff foam jacket.

19. The method according to claim 18, further including:
applying end caps to each end of the foam jacket which provide strain relief to the electrically conductive wires.

20. (canceled)
